(19) United States
(12) Patent Application Publication
(10) Pub. No.: US 2010/0099076 A1
(43) Pub. Date: Apr. 22, 2010

(54) SENSITIVE AND RAPID DETECTION OF VIRAL PARTICLES IN EARLY VIRAL INFECTION BY LASER TWEEZERS

(75) Inventors: Hanbin Mao, Kent, OH (US); Hanwen Mao, Annandale, VA (US); Zian Mao, HangZhou (CN); Jiangsen Mao, HangZhou (CN)

Correspondence Address: HAHN LOESER & PARKS, LLP, One GOJO Plaza, Suite 300, AKRON, OH, 44311-1076, US

(73) Assignee: Kent State University, Kent, OH (US)

(21) Appl. No.: 12/252,623

(22) Filed: Oct. 16, 2008

(51) Int. Cl. C12Q 1/70; C12M 1/00

(52) U.S. Cl. 435/5; 435/287.1; 435/288.7

(57) ABSTRACT

The present system and methods allow for low level detection of as little as single pathogen particles, such as viral or bacterial particles, during the early stage of infection. An optical trapping system, such as laser tweezers, are used to trap a substrate to which an analyte has been bound to detect and record the thermal motion of an antibody-antigen interaction that may occur between an anti-viral antibody-coated microsphere and a viral particle for example. The system may be equipped with a detection system such as a position sensitive photodetector (PSD) to record the thermal motion of a trapped microsphere and particle at a certain frequency. The thermal motion data may be Fourier transformed into a power spectrum, which may be transformed into an output value using a Lorentzian equation. The power spectrum of the trapped microsphere may be recorded before and after binding of the pathogenic particle to determine the presence thereof.

### BACKGROUND AND SUMMARY

The present disclosure relates to a system and method for detecting analytes in a medium and more particularly to method for detecting low levels of analytes such as antigens including viruses or other microbes and pathogens.

In the case of viral infection, the infection starts with an initial peak of what is called “viremia” a few days after infection. Viremia is the medical condition where viruses enter the bloodstream and hence have access to the rest of the body. Viremia is followed by the decrease of the viral level as a result of the activation of a person's immune system. The immune system interacts with the virus to either completely control viral infection, partially control viral infection, or not control viral infection at all which leads to increase in virus levels. The initial peak of viremia generally occurs within one or two weeks after infection. This initial peak of virus levels gives medical professionals a window for possible detection of viral copies and rapid diagnosis of viral diseases.

With viruses, bacterium or other microbes or pathogens, an antibody is a molecule produced by the immune system of a human or an animal in response to a foreign particle or pathogen. The antibody is able to chemically bond to a particular portion of the foreign particle known as the antigen. A foreign particle may have several antigens, though a particular antibody binds to only one of them. The recognition and subsequent binding of the antibody are among the initial stages in the immune response, and specific antibodies are produced by the body in response to particular pathogens. Therefore, the presence of a particular antibody in the blood is an indicator of a particular infection, which may be found before the onset of any signs or symptoms of the disease. In general, it has been the approach to look for the presence of the antibody as an indicator of disease rather than to detect the pathogen directly as the antibody levels in the blood generally far exceed the pathogen levels. After viral infection for example, a person's immune system will start to produce antiviral antibodies. Viral specific antibodies are usually generated within several weeks after infection. A medical professional can accurately diagnose a patient with a possible viral infection by examining the interaction of the anti-viral antibodies. However, levels of antibody may only become detectable using known techniques when the controllable stage of the viral disease already passes.

Also, due to the strong chemical bonds between a particular antigen found on the surface of the pathogen and an antibody, particular antigens can be isolated and used to detect the presence of antibodies and thus the presence of a pathogen in the body. In such an approach, a sample of blood is exposed to an isolated antigen, and if the blood sample contains antibody specific for that antigen, then it will chemically bind to the antigen. Detection of such binding can be performed in various ways, such as by precipitation, direct and indirect immunofluorescence and immunoassay techniques.

Various approaches to detecting infection have been developed, including detection methods performed at a later stage of infection, with detection of antigens and/or antibodies produced against the microbe or virus or through the identification of the DNA/RNA from these microbes after PCR/RT-PCR amplification.

Early treatment of an infectious disease, such as that caused by a virus, bacterium or other pathogen, can provide an efficient therapy and immediate control of a virus (or other microbe) outbreak. A rapid and accurate diagnosis of infection at the early stage of infection provides many benefits. There are currently three categories of diagnostic methods: microscopic diagnosis, molecular diagnosis, and serologic diagnosis (which aims to detect the level of antibody against specific pathogens such as virus and microbes as described above). In microscopic diagnosis, individual viral particles can be observed with electron microscopes. The microscopic detection of organisms stained with fluorescent dyes or other markers attached to antibodies has been developed for the specific identification of some viruses. Although useful, fluorescent dyes will be destroyed under prolonged exposure of the excitation light (where absorption is maximal) due to photoactivated chemical reactions, causing bleaching, and inhibiting proper detection. Such a problem makes the use of fluorescent dyes problematic in attempting to detect small numbers of or single virus particles, or other pathogens or microbes. Since only a few molecules of fluorescent dye can be attached to the single virus, which is usually very small (hundreds of nanometers and below), proper detection is inhibited. Due to the small amount of the dyes, bleaching only a few of them can cause the dramatic decrease of the signal and even total loss of the signal.

In molecular diagnosis, viral DNA, RNA, or proteins from a clinical sample can be used to identify the infectious agent. Diagnosis based on immunoassays, such as enzyme-linked immunosorbent assay (ELISA), enzyme immunoassay (EIA), and radioimmunoassay (RIA), are known for the detection of antigens. The basic principle in many of these assays is that an enzyme-, chromogen-, fluorogen-, radionucleotide-, or nanoparticle-conjugated antibody permits antigen detection upon antibody binding. In order for this interaction to be detected as a color, fluorescence, or radioactivity change, significant numbers of antibodies must be bound to a correspondingly large number of antigen epitopes. Rapid and accurate diagnosis of a viral or other infection is critical for the prompt provision of anti-viral or other therapy, timely cure of the disease, and immediate control of a possible outbreak. This is especially true when pathogens are highly contagious and no treatment is available for those potentially employed by bioterrorists.

Thus, there is a need for a system that rapidly, reliably, and automatically detects viral or other microbial or pathogenic particles, especially when present in very small quantities, as in the early stage of an infection, and consequently provides a measurable signal in near real time conditions. Currently, the methods available test the presence of anti-bodies at a later stage of an infection when symptoms have already started to develop in an individual. At this stage, it is often too late to prevent the spread of infection or an outbreak in the population. The apparatus, method, and system of the present invention is suitable for a variety of applications: chemistry, biochemistry, immunology, etc. Applications for which the present invention are presently suited are immunological assays (“immunoassay”). Such techniques are directed to, for example, probing antigen-antibody interactions.

The present disclosure relates to systems and methods that allow for highly sensitive and simple detection method of viral and other microbe particles during the initial peak of viremia or infection. The system and methods may utilize optical tweezers to provide a rapid and accurate detection method to identify the infection. By using focused laser beams, optical tweezers can trap and remotely manipulate dielectric particles. The particles may include cells, bacterial and viral particles. The embodiments of the apparatus and methods of the present invention addresses the deficiencies of prior systems and methods, and allow a small reaction substrate formed at least one nano- or micro-particle to be used in detecting as little as a single microbe with high sensitivity. The systems and methods provide for detection of a microbe or other analyte which can be effectively used in various environments, including where the virus, bacterium or the like is released, such as for combating bioterrorism in field environments, as in the case of bioterrorism for example. The invention simplifies use and detection at a very early stage of infection, and can be used to detect multiple analytes or microbes.

The presently disclosed systems and methods for rapid detection of analytes comprises a chamber having at least one channel. A plurality of nano- or micro-particles, such as microspheres, are provided and have a coating comprising a binding agent, such as a protein binding agent. A sample having one or more analytes capable of binding to the microspheres are presented into proximity of the microspheres, and a trapping means capable of trapping at least one of the plurality of microspheres is used to isolate at least one microsphere. A detection system for detecting the thermal motion of at least one of the plurality of microspheres is provided, and a control system for determining the presence of a viral or other microbe particle in the sample is provided.

In an embodiment, the systems and methods enable determining the presence of an analyte comprising the steps of coating a plurality of nano- or micro-particles, such as microspheres, with a binding agent, and mixing the plurality of microspheres with an antibody to form a sample. A system including a chamber having at least one channel is provided, and the sample is loaded. Trapping of at least one of the plurality of microspheres is performed with a trapping system. Thereafter, flowing a medium containing one or more analytes through the chamber is performed, and detecting the thermal motion of at least one of the trapped microspheres with a detection system by recording a power spectra of at least one of the trapped plurality of microspheres. From the power spectra, determination of the presence of one or more analytes is provided.

The foregoing and other aspects will become apparent from the following detailed description when considered in conjunction with the accompanying drawing figures.

### BRIEF DESCRIPTION OF THE DRAWINGS

FIG. 1 is a schematic diagram of the apparatus according to an example of a system and method for rapid detection of an analyte or antigen;

FIG. 2 is a schematic diagram of an example microfluidic chamber usable in the systems and methods described in examples;

FIG. 3 is a graph of the data obtained from the system and method;

FIG. 4 is a schematic diagram of the microsphere and antibody-antigen interaction.

### DETAILED DESCRIPTION OF THE DRAWINGS

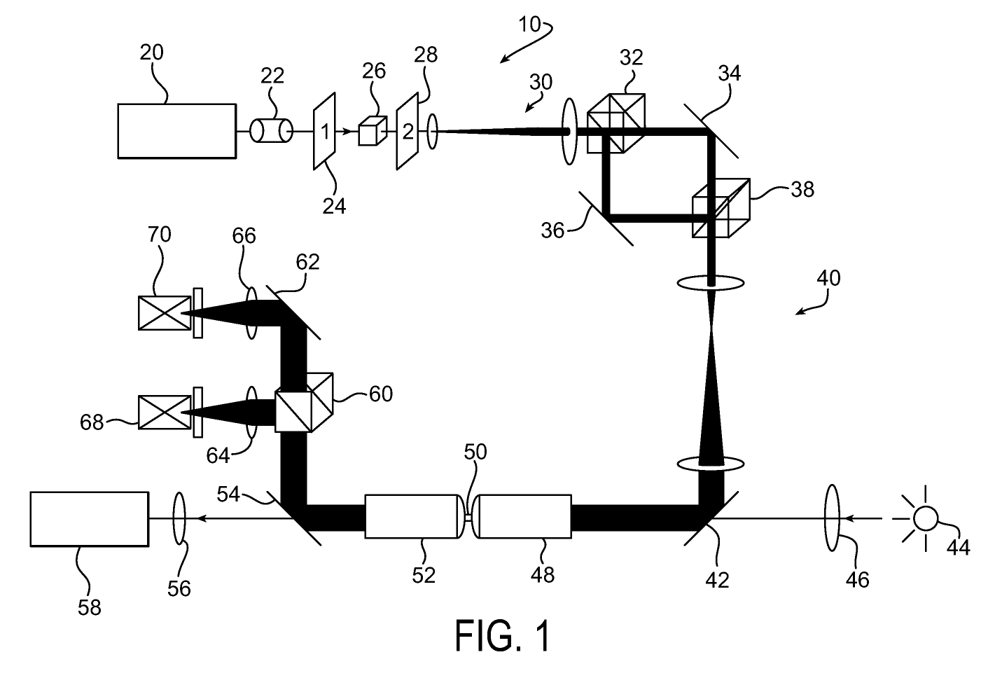

Systems and methods according to embodiments to provide for rapid and reliable detection of microbes will be described with reference to FIGS. 1-4. The system and methods are designed for use in field environments for example, to allow for the rapid and accurate diagnosis of early viral or other microbe infection, to allow for rapid anti-viral or other therapy, and prompt control of a possible outbreak. This may be important when pathogens are highly contagious and/or no treatment is available, and may be useful for bioterrorism response. In the case of viral infection for example, early treatment can provide for an efficient therapy and control of a virus outbreak. Although in theory, the window of peak viremia at the initial stage of an infection can be utilized in detection, it is often transient, unpredictable, and variable among different viruses. Traditional approaches may not have sufficient sensitivity, and can generate false negative results, and may also require time-intensive and tedious procedures. For example, in a PCR or ELISA techniques as mentioned, attempting to detect the presence of antibodies in ELISA, samples must be purified, and the choice of optimal probes for hybridization and primers for PCR is quite challenging, particularly where the virus has high rates of genetic mutation.

In the embodiments shown, the system may use an optical trapping system 10 such as comprising a laser-tweezers arrangement shown in FIG. 1 The optical trapping system 10 captures at least one particle from solution, and can manipulate the particle to facilitate detection. By using focused laser beams, optical tweezers can trap and remotely manipulate dielectric particles. These particles include cells, bacterial and viral particles, and other microbes. In the embodiments, the binding of antibody-antigen is used to detect the presence of a pathogen, and the counterpart is used as bait. The optical trapping system 10 allows for delicate detection and recording of the antibody-antigen interaction between an anti-viral-antibody-coated substrate and viral particles for example. The substrate may be any nano or micro sized particle, such as a micro or nano-sized polystyrene bead for example.

FIG. 1 depicts various features of the optical trapping system, to capture, hold, and/or transfer a selected particle by a three-dimensional restrictive force. This force is ideally suited to hold micro or nano-sized dielectric particles at its beam focus in a liquid medium. In the system as shown in FIG. 1, an incident laser beam is provided by a laser source 20, which may have any suitable wavelength, such as a wavelength that is well separated from the excitation and emission bands of the materials to be detected. Generally, a laser having a wavelength of >650 nm may be used, such as a 1064 nm YAG laser for example. The laser beam may be directed to a Faraday isolater 22 which is an optical component that allows the transmission of light in only one direction to prevent unwanted feedback of laser light. The laser is set up so that its beam passes through a Faraday isolator 22 to eliminate back reflection from the downstream optical path. The Faraday isolator 22 increases the stability of the laser output, which assists in highly sensitive position measurement. A half wave plate 24 is then provided which retards one polarization by half a wavelength, or 180 degrees. This type of wave plate rotates the polarization direction of linear polarized light. A polarized beam splitter 26 splits the beam, and provides a polarized beam to the objective lens of a first telescope 30. The polarized light is split into “S” polarized light and “P” polarized light. The “S” polarized light is reflected to an optical dump and the “P” polarized light is propagated through a second half wave plate 28 to readjust polarization. Expanded light from the telescope 30 is directed to a polarized beam splitter 32, which splits the beam to direct a “P” polarized component to a reflecting mirror 34 and a “S” polarized component to a steerable mirror 36, with each component reflected back to a polarized beam splitter 38 to combine the two beams. A second telescope 40 further expands the combined light and directs it to a dichroic mirror 42 where it is combined with a blue light from a source focused through a lens 46 and directed to a first objective 48 and through a sample cell 50 to a second objective 52. The light is then directed to a dichroic mirror 54, with the blue light portion passing to a lens 56 and focused to a video camera 58. The laser light is reflected by dichroic mirror 54 to a polarized beam splitter 60 which splits the beam such that a portion is directed to mirror 62. The split laser beams pass through first and second focusing lenses 64 and 66 and to first and second position sensitive photodetectors 68 and 70, respectively. In an example of the system, the light is expanded by the two telescopes 30 and 40 to reach a final diameter of 12 mm (1/e²). During the two-stage expansion and collimation, the laser light is split by the polarized beam splitter 60 into two beams of “P” and “S” polarized lights. The “S” polarized light is controlled by the mirror 36 at a conjugate plane of the back focal plane of the focusing objective 48. As described above, the “S” polarized light and “P” polarized light are then combined by a third polarized beam splitter 38 and directed toward an objective 48. The objective 48 then focuses the light into a sample cell 50, which as will be described hereafter, may be a microfluidic chamber. The “P” and “S” polarized light are collected by a second objective 52 as they exit the microfluidic chamber 50. The polarized light beams are split according to “P” or “S” beams at 60. The detecting means detects the polarized light beams upon exit from the microfluidic chamber 50. As shown in FIG. 1, the detecting means suitable for detecting thermal motion of one or more trapped particles are position sensitive photodetectors (PSD) (DL100, Pacific Silicon Sensor, Westlake Village, Calif.), quadrant photodiode/detectors or other suitable detectors.

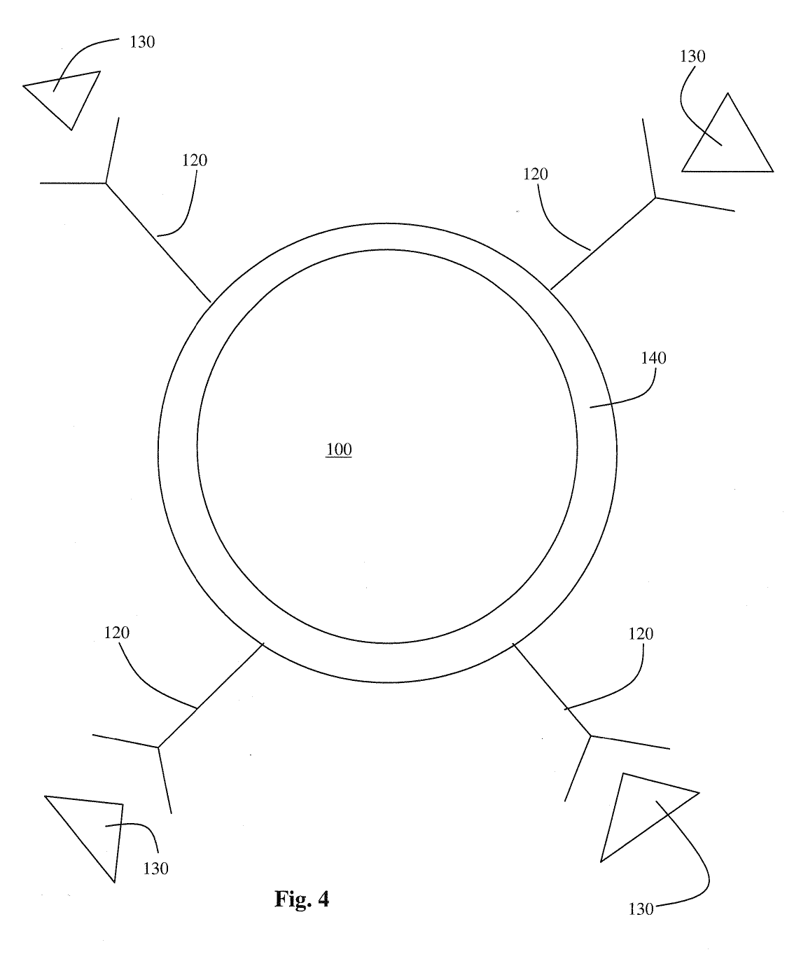

The optical trapping system focuses a laser beam through the solution in sample cell 50 to capture a dielectric particle, such as a microbead, at the beam focus. The particle is pulled into and held stable in the trap created by the beam. The trapped particle can then be observed within the sample cell and remain fixed during reaction and detection. As an example, the optical trapping system 10 may be mounted relative to a reaction chamber, such that the laser beam is directed through the objective lens 48, and into the solution where the particles reside, with at least one particle being trapped as desired. The optical trapping system 10 may remain fixed in place, and movement of the particle performed by flow into the reaction chamber or physical movement thereof. The detection system is comprised of the position sensitive photodetectors to record the antibody-antigen interaction between an antibody coated microspheres. As seen in FIG. 4, the antibody coated microspheres 100 bind to an analyte 130 or an antigen, such as a virus such as the human immunodeficiency virus (HIV) or other microbes and pathogens, for detection thereof. The position sensitive photodetectors (PSD) 68 and 70 record the thermal motion of a trapped microsphere and particle at a certain frequency. The thermal motion data may be Fourier transformed into a power spectrum, which may be transformed into an output value using a Lorentzian equation. The power spectrum of the trapped microspheres may be recorded before and after introduction of the antigen or analyte to the sample containing the microspheres to determine the presence of the antigen or analyte.

The systems and methods of the invention allow detection of even a single pathogen, or corresponding antigen or analyte in a simplified and convenient manner. As compared to methods where fluorescence is used in detection of the binding of between an antibody or antigen and the pathogen/microbe, detection of a single or very small number of microbes is difficult because the fluorescent signal is very small. In order to detect such a signal, a highly sensitive detection method would be needed. Such high sensitivity requirements would add significantly to the cost and complexity of the detection system. And even with the high sensitivity fluorescence detection method, the fluorescence bleaching cannot be avoided, which will yield false negative results. In the present system, the ability to detect changes in thermal motion of a trapped microsphere is provided without higher cost and complexity. Further, the use of fluorescence requires additional preparation of fluorescence materials, and the involvement of fluorescence material may change the property of the entity to be detected. For example, an antibody labeled with a fluorescent dye may have reduced avidity towards a corresponding antigen. The systems set forth herein do not require fluorescing materials and avoid such problems. The use of fluorescence may also require additional antibodies to be used in the detection method. In a fluorescence scheme, the binding of antibodies to a virus or other microbe may further require the use of a second or further types of antibodies used to also bind to the antibody-virus complex in a so called sandwich assay to yield a new complex: antibody-virus-antibody-secondary antibody. This secondary antibody is either labeled with fluorescence or with an enzyme. In the latter case, the enzyme will convert a non-fluorescent or non-chromogenic substrate to a fluorescent or chromogenic product, which may then be detected. In the present approach, the binding of the virus is detected directly by thermal motion, and no other antibody or further steps are required. As a comparison, the detectable complex in the present method is, substrate-antibody-virus, which is simplified and reduces the material (ie, antibody) cost, and also simplifies the procedures for detection, making it possible to perform a more rapid assay in environments such as hospitals, clinics, and labs or in the field.

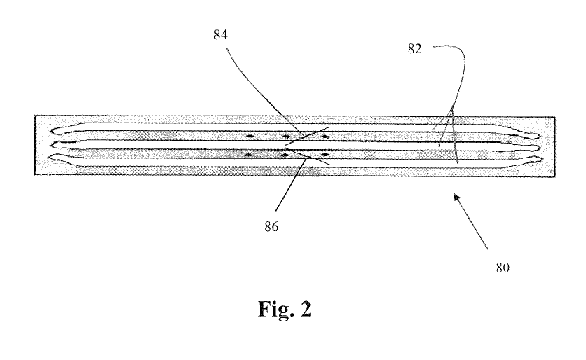

The apparatus may include a microfluidic chamber 80, as shown in FIG. 2. The microfluidic chamber 80 may be positioned between the objectives 48 and 52 as shown in FIG. 1. The chamber 80 as shown in FIG. 2 may be constructed from Nescofilm (manufactured by Azwell, Osaka, Japan) and glass cover slips. The chamber 80 may be prepared by sandwiching a piece of the Nescofilm between two glass cover slips. At least two of the microchannels 82 may be imprinted into the Nescofilm by a laser cutter. The channels 82 are then sealed through thermal bonding. The channels in the chamber 80 may function as either a main channel or as a service channel for solutions. The channels 82 may be connected through a microcapillary tube 84 and 86 for example. In the present embodiment, the microcapillary tubes 84 and 86 are about 20 μm i.d. for example. The direction of flow inside the microcapillary tube may be controlled by pressure. The channels may have different pressures so that the differential in pressure causes the microspheres 100 to flow from the channel with higher pressure to the channel with lower pressure.

Each microchannel 82 in the chamber 80 may have a width of about 2 millimeters and a length of about 4 centimeters, but the dimensions are not limited to this particular embodiment and may be modified according to the desired purpose. For example, size can be reduced to 20 μm or other suitable dimensions to reduce the dead volume of the system and accelerate detection speed. Each microchannel 82 may have an inlet and outlet for the insertion and removal of the solution or sample. In the present embodiment, the inlet/outlet may be 2 mm in diameter, but are not limited to this configuration and may be adjusted according to the dimensions of the channels or other considerations. The chamber 80 may also contain one or more thermocouples to record the temperature inside the microchannels. In this example, the top and bottom channels 82 may be used for housing a plurality of nano or micro particles which have been coated with a binding agent for binding with a microbe, antibody, antigen or other analyte. The center channel 82 may then be used as a detection channel into which one or more coated particles are selectively introduced. Once one or more coated particles are trapped in the center channel 82, a sample containing a suspected virus or other microbe may be introduced to the center channel 82 and flowed past the trapped particle, wherein the binding thereof to the coated particle can occur and be detected. Other configurations are possible and contemplated to not only isolate the one or more particles, but to allow introduction of a sample (blood or water for example) for binding of the analyte to be detected thereto and detection of the analyte according to the methods described herein.

The nano or micro-particles 100 shown in FIG. 4 may be constructed from at least one of the group consisting of polystyrene, glass, silica, mica, ceramic or other dielectric polymers and dielectric materials. In the example shown, the particles are spherical, but need not be. In the example, the diameter of the microspheres 100 may be adjusted according to the desired purpose of the user and is not limited to any particular dimension. In the present embodiment, the microspheres 100 may have a diameter in the range between about 200 nanometers to 1 micrometer. The microspheres 100 may also have a diameter as low as 40 nm as long as the diameter of the microspheres 100 is equal to or greater than the diameter of the particle. The microspheres 100 may have an index of refraction of 1.3 or greater. Any material may be used for the microspheres 100 as long as the index of refraction is greater than the index of refraction for an analyte containing solution, which is 1.3 for a solution with water as a major component.

In operation, the substrates such as microspheres 100 may be coated with a binding protein agent 140, such as, but not limited to streptavidin. Next, the protein coated microspheres 100 may be mixed with various antibodies 120 to create samples according to the following embodiments.

### EXAMPLE 1

The plurality of microspheres 100 are coated with a protein binding agent 140, for example, streptavidin. The plurality of microspheres 100 in this example are about 970 nm in diameter. The protein coated microspheres 100 are then mixed with anti-virus antibody to create a sample. In this example, the anti-virus antibody is anti HIV antibody (rabbit anti-P24 Immunoglobulin G (IgG). The sample may be incubated for 20 to 30 minutes at room temperature and then centrifuged. After centrifuging, the sample is mixed with a buffer solution and mixed thoroughly.

### EXAMPLE 2

The second example according to the presently disclosed system and method may be used as a control sample. The second sample may be made by coating the plurality of microspheres 100 with a protein binding agent 140 such as streptavidin. Next, the protein coated microspheres 100 are mixed with a control antibody. In the present embodiment, the control antibody is a buffer solution containing rabbit inmunoglobulin G (IgG) control antibody. The second sample may be incubated for 20 to 30 minutes at room temperature and then centrifuged. After centrifuging, the sample is mixed with a buffer solution and mixed thoroughly.

According to the presently disclosed system and method, a solution containing the analyte 130 to be detected, such as an antigen or a virus, may be prepared or acquired from environmental or human medium, for example, water, or human blood. In the present embodiment, the anlayte is an HIV virus or a virus like particle (VLP, Functional Genetics, Inc.). The virus like particle may be used instead of actual HIV virus because of the low biohazard level requirement of the virus like particles. The virus like particles consist of viral proteins derived from the structural proteins of the virus embedded within a lipid bilayer derived from infected cells. These particles resemble the virus from which they were derived but lack viral nucleic acid, which means that the virus like particles are not infectious.

After preparing the samples, the optical trapping system 10 is set up according to FIG. 1 so that the microfluidic chamber 80 is positioned between the objectives to form a laser trap. The laser tweezers 10 are set up in a two trap configuration with a distance of 10 μm between the two laser traps in the Y-direction as shown in FIG. 1. A buffer solution may be flowed through one of the channels in the chamber 80 at a rate of about 5 μL/min for example.

The first sample containing the microspheres 100 is then loaded into one of the channels 82 (top or bottom) of the microfluidic chamber 80. The sample may be flowed through the channel at a rate of between about 1 μL/min to 5 μL/min. Other suitable flow rates are contemplated, such as between flow rates, for example, from 1 pL/min to 1 mL/min). As the first sample containing the microspheres 100 are flowed through the center channel of the chamber 80, the laser tweezers trap at least one microsphere.

The second sample containing another type of microspheres 100, microspheres coated with an control antibody but not the anti-antigen antibody described in the preceding paragraph for example, may be flowed through a second channel 82 (bottom or top) of the chamber 80. The second sample may be flowed through the channel at a rate of between about 1 μL/min to 5 μL/min. Other suitable flow rates are contemplated, such as between flow rates, for example, from 1 pL/min to 1 mL/min). After the second sample is flowed through the center channel of the chamber 80, the laser tweezers trap at least one microsphere, and the rate of flow in the second channel (bottom or top) of the chamber 80 is reduced to about 1.5 μL/min. The center channel of the chamber 80 is then loaded with the third sample containing the analyte 130 to be detected. As shown in FIG. 4, the antibody 120 will bind to the protein coated microsphere 100 and then the antigen 130 will bind to the corresponding anti-antigen antibody 120. For the laser tweezers trapped microsphere coated with the control antibody, no binding of antigen will occur.

After all the samples have been loaded and flowed through the chamber 80, the power spectra of the trapped microspheres 100 are recorded every 10 minutes for the first 30 minutes, every 1 minute for the next 30 minutes, every 30 seconds for the next 20 minutes, and every 1 minute for an additional 10 minutes. During this time, the thermal motion of the trapped microspheres 100 may be recorded at 100 kHz for 0.6 second time intervals by the position sensitive photodetector. The recording step is not limited to this particular time intervals and may be adjusted according to the desired purpose by one of skill in the art.

After recording the power spectra of the trapped microspheres 100, the power spectra are fit with a Lorentzian equation shown below:

$${( Δ {F}^{2} ( ω ) )}_{eq} = 4 ξ {k}_{B} T \frac{{ω}_{c}^{2}}{( {ω}^{2} + {ω}_{c}^{2} )}$$

The power spectrum is fit using the equation above with an Igor Pro 5 (WaveMetrics, Lake Oswego, Oreg.) program. In the Lorentzian equation, {ΔF²(ω)}_{eq} is the equilibrium spectral density of force fluctuations (in units of force squared per frequency) exerted on the trapped particle, ξ is the drag coefficient, k_{B} is the Boltzmann constant, ω is the angular frequency, T is absolute temperature, and the corner frequency, ω_{c}, can be described as the following:

$${ω}_{c} = \frac{{k}_{stiff}}{ξ}$$

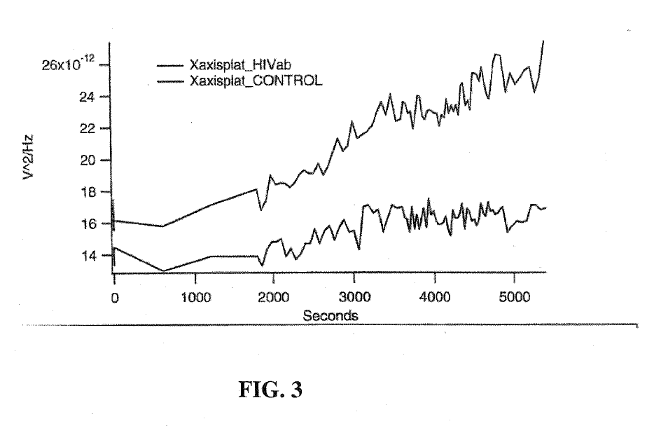

With reference to FIG. 3, the plateaus of the power spectra from the thermal motion of the trapped microspheres 100 may be recorded against time. The plateau value and corner frequency of the power spectrum will depend on the size and refractive index of the trapped microspheres 100. We have found that a larger plateau value and corner frequency indicates a larger size of the microspheres 100. The increase or decrease of plateau values and corner frequencies in FIG. 3 may indicate that one or more of the analytes has become bound to the microspheres 100.

The system and methods also provide for a non-intrusive or in situ detection approach. For example, where a blood sample is used in the detection approach, the sample can be reused for other purposes if desired, as it incurs no contamination. The microspheres or other particles used can be filtered from the sample after detection. Alternatively, microspheres or other particles other than those trapped can be washed away before the blood sample is introduced; while the trapped microspheres or other particles can be kept trapped until all of the blood sample is analyzed and recollected. In the microfluidic chamber 80 shown in the example, the service channels 82 at the top and bottom may be used to hold the microspheres or beads coated with antibodies. These channels 82 are connected through the center (assay) channel via micropipettes 84 and 86 as described. The direction of the flow inside the micropipettes 84 and 86 is precisely controlled by gravity and/or pressure. To trap a bead, a flow is directed from a service channel 82 at the top or bottom to the center channel 82 so that microbeads are available in the center channel 82. Once the bead coated with the antibody is trapped by laser tweezers, the flow is directed from center channel 82 to a service channel 82 so that free beads flow back to the service channel 82, and the buffer in the service channel therefore doesn't or wouldn't contaminate the blood sample introduced into the center channel 82. Therefore, apart from the front end of blood sample that may be mixed with buffers in the service channels, the majority of the blood sample is free of contamination except its exposure to the trapped bead or beads (typically one or two for example). Considering the small size of these trapped beads (1 micrometer in diameter for example), this exposure will not change the property of the blood sample. In fact, the small service area of the trapped beads makes it possible to perform the detection of a virus or other microbe using alternative methods on the recollected blood sample, even if the virus titration is very low in the original sample (since only a few virus particles are consumed by the trapped bead(s)). This possibility can thus provide alternative purposes, such as enabling another detection method to be used on the virtually same (blood) sample so that results from the method can be confirmed. The approach of using two or more detection methods to verify a virus or other infection may be desirable. Further, the approach allows for detection of very small number of virus or other microbe particles to detect the infection at a very early state, which is desirable for prognosis in a quick manner.

As the microspheres 100 are flowed through the chamber 80, the microspheres 100 also undergo hydrodynamic coupling. Hydrodynamic coupling occurs when the distance between two trapped particles is close to the sum of their diameters. The hydrodynamic coupling may cause two microspheres 100 to rotate in different directions under a laminar flow. The rotation of trapped microspheres 100 may allow a survey of the entire or at least a majority of the surface area of the microspheres 100, thereby enhancing the detection of a single or small number of microbes bound thereto. The survey may be used to determine multiple viral or other particles binding to the microsphere 100. It is desirable for the quantitation of virus or other microbe or pathogen concentration, for example.

The surface area of the microspheres 100 and particles inside the microchamber may be imaged by a CCD video camera (ie, NT39-244, Edmund Optics, Barrington, N.J.), shown as 58 in FIG. 1, under the illumination of a blue LED bulb (ie, P465-ND, Digikey, Thief River Falls, Minn.) shown as 44 in FIG. 1. The CCD video camera 58 may allow for a visual survey of the surface of the microspheres 100 depending upon the size of the microspheres 100, and may allow for the detection of the particle being attached to the microspheres 100 depending on the particle size.

The presently disclosed apparatus and method may also be used with a configuration of multiple laser tweezers 10. For example, two laser tweezers 10, with different wavelengths of trapping lasers, may be positioned in the arrangement of FIG. 1 and directed towards the microfluidic chamber 80. With multiple laser tweezers 10, it may be possible to detect different viral particles in a sample by flowing the samples into separate chambers. The microfluidic chamber 80 may therefore be configured in other suitable manners to provide for detection of multiple microbes in a single sample for example. Alternatively, an acousto-optic modulator or an electro-optic modulator can be used to modulate the laser beam(s) at about MHz frequency, which can generate multiple traps for detection of multiple microbes.

It will be readily apparent to those skilled in the art that by use of these techniques, it is possible to detect and quantitate any and all antigens, antibodies and other analytes at extremely low concentrations. The above examples are intended to be illustrative rather than limiting. Those skilled in the art who have reviewed this specification will readily appreciate that the techniques described herein can be adapted to detection and quantitation of other analytes without departing from the scope of the present invention.

All of the methods and apparatus disclosed and claimed herein can be made and executed without undue experimentation in light of the present disclosure. It will be apparent to those of skill in the art that variations may be applied to the methods and apparatus described herein without departing from the concept, spirit and scope of the claimed subject matter. More specifically, it will be apparent that certain agents that are both chemically and physiologically related may be substituted for the agents described herein while the same or similar results would be achieved. All such similar substitutions and modifications apparent to those skilled in the art are deemed to be within the spirit, scope and content of the claimed subject matter.

What is claimed is:

1. A system for sensitive and rapid detection of analytes comprising:
a chamber having at least one channel,
a plurality of microspheres having a coating comprising a protein binding agent, wherein the microspheres are mixed with an antibody to form a sample,
one or more analytes capable of binding to at least one of the plurality of microspheres,
a trapping means capable of trapping at least one of the plurality of microspheres,
a detection means for detecting the thermal motion of at least one of the plurality of microspheres,
a control unit for determining the presence of an analyte attached to the microspheres.

2. The system according to claim 1, wherein the microspheres are constructed from a material selecting from the group consisting of polystyrene, glass, ceramic, mica or other dielectric materials.

3. The system according to claim 1, wherein the microspheres have a diameter in the range between about 40 nanometers and 20 micrometer.

4. The system according to claim 1, wherein the trapping means is an optical laser having a beam of about 1064 nm.

5. The system according to claim 1, wherein the detecting means is at least one position sensitive photodetector or quadrant photodiode/detector.

6. The system according to claim 1, wherein the analyte is a pathogen, antigen or a virus.

7. The system according to claim 1, wherein the microspheres have an index of refraction equal to or greater than 1.3.

8. A method for determining the presence of a viral particle comprising the steps of:
coating a plurality of microspheres with a binding agent,
mixing the plurality of microspheres with an antibody to form a sample,
providing a chamber having at least one channel,
loading the sample into the chamber,
flowing the sample through at least one of the channels in the chamber,
trapping at least one of the plurality of microspheres with a trapping system,
flowing a solution containing one or more analytes into the chamber,
detecting the thermal motion of at least one of the microspheres with a detection means by recording a power spectra of at least one of the plurality of microspheres, and
determining the presence of the one or more anlaytes.

9. The method according to claim 8, wherein the plurality of microspheres are constructed from a material selecting from the group consisting of polystyrene, glass, ceramic, mica or other dielectric materials.

10. The method according to claim 8, wherein the plurality of microspheres are in the range between about 40 nanometers to 20 micrometer in diameter.

11. The method according to claim 8, further comprising the step of Fourier transforming the thermal motion using a Lorenztian equation.

12. The method according to claim 8, wherein the trapping means is a laser having a beam of approximately 1064 nm.

13. The method according to claim 8, wherein the binding agent is a protein binding agent.

14. The method according to claim 8, wherein the detection means is at least one position sensitive photodetector or quadrant photodiode/detector.

15. The method according to claim 8, wherein the analyte is a pathogen, antigen or a virus.

16. The method according to claim 8, wherein the rate of flow through the chamber is between the range of approximately 1 pL/min to 1 mL/min).

17. The method according to claim 8, further comprising the step of mixing the microspheres with a control antibody to form a second sample.

18. The method according to claim 8, further comprising the step of flowing the second sample through the chamber at a rate of between the range of approximately 1 pL/min to 1 mL/min).

19. The method according to claim 8, wherein the rate of flow through the chamber is reduced to about 1.5 μL/min.

20. The method according to claim 8, further comprising the step of recording the power spectra at a rate of 100 kHz for 0.6 s.
